(19) United States
(12) Patent Application Publication
(10) Pub. No.: US 2007/0254914 A1
(43) Pub. Date: Nov. 1, 2007

(54) Low-concentration atropine solution for preventing myopia progression and preparing method thereof

(75) Inventors: Pei-Chang Wu, Niaosong Township (TW); Hsi-Kung Kuo, Niaosong Township (TW); Po-Chiung Fang, Niaosong Township (TW); Jong-Jer Lee, Niaosong Township (TW); Chih-Hsin Chen, Niaosong Township (TW)

Correspondence Address: NIKOLAI & MERSEREAU, P.A., 900 SECOND AVENUE SOUTH, SUITE 820, MINNEAPOLIS, MN, 55402, US

(73) Assignee: Non-Profit Organization Chang Gung Memorial Hospital, Gueishan Township (TW)

(21) Appl. No.: 11/415,256

(22) Filed: May 1, 2006

(51) Int. Cl. A61K 31/46

(52) U.S. Cl. 514/304

(57) ABSTRACT

A low-concentration atropine solution for preventing myopia progression contains an atropine concentration less than 0.1% (w/w). Preferably, the atropine concentration is 0.05% (w/w) in optimal situation. The low-concentration atropine solution in treatment causes less photophobia and systemic side-effects to patients and has excellent compliance to reduce damages from ultraviolet and hazard blue light and to avoid visual morbidities such as cataract and retinal macula lutea deterioration.

### BACKGROUND OF THE INVENTION

1. Field of the Invention

The present invention relates to a low-concentration atropine solution for preventing myopia progression, and more particularly to a low-concentration atropine solution substantially relative to human medication to prevent exacerbation in myopia progression.

2. Description of Related Art

Myopia is a crucial ophthalmic morbidity, especially with high myopic diopter, that causes many complications such as youth cataract, glaucoma, retinal detachment, macular hemorrhage, and retinal degeneration. In Taiwan, myopia is the second factor which causes blindness in aged people so that prevention for myopia progression is important. Preventing high myopia has to be undertaken since childhood. Asian children suffer myopia increase 75 to 100 degree (0.75 to 1 Diopter) per year and western children the same increase 50 degree (0.5 Diopter) per year. Once children have myopia, the myopic degrees continuously increase until the end of adolescence so that those children are in great danger to suffer high degree myopia. Therefore, efficiently preventing myopia progression in children is an important issue.

With regard to existed researches and assays studying on ways to prevent myopia progression and avoid high degree myopia, all indicate that the atropine is the only efficient medicine to inhibit myopia progression for schoolchildren.

In a study of “Effects of Different Concentrations of Atropine on Controlling Myopia in Myopic Children” (Shih Y F, Chen C H, Chou A C, et al. J Ocul Pharmacol Ther 1999;15:85-90., cited as “Shin's study” in the following description), 0.5% atropine solution is most efficient. Although powerful 1.0% atropine solution is used in many countries and 0.5% atropine solution is commonly used in Taiwan, those solutions still have some drawbacks in clinic application because patients will have photophobia that disorders their daily outdoor activities. Therefore, the compliance is poor and dropout rate is correspondingly high.

### SUMMARY OF THE INVENTION

To overcome the drawbacks of the conventional atropine solution for preventing myopia progression, a modified low-concentration atropine solution is provided to eliminate the drawbacks.

A main objective of the present invention is to provide a low-concentration atropine solution for preventing myopia progression that has excellent preventing efficiency for myopia progression with less side-effects.

Another main objective of the present invention is to provide a method for preparing the foregoing atropine solution.

To achieve the foregoing objectives, the low-concentration atropine solution for preventing myopia progression comprises:

atropine concentration less than 0.1% (w/w).

Preferably, the atropine concentration is 0.05% (w/w).

The preparing method for the low concentration atropine solution is to dilute an atropine ingredient to obtain the low-concentration atropine solution having atropine concentration less than 0.1(w/w).

Further benefits and advantages of the present invention will become apparent after a careful reading of the detailed description.

### DETAILED DESCRIPTION OF THE PREFERRED EMBODIMENT

A low-concentration atropine solution for preventing myopia progression in accordance with the present invention comprises atropine concentration less than 0.1% (w/w).

As described in the foregoing description, the efficient administration of atropine solution is surely regarded to contain 0.5% (w/w) atropine concentration and never assumed to contain the atropine concentration less than 0.1% (w/w).

In the present invention, the lower-concentration atropine solution for preventing myopia progression is defined to have the atropine concentration less than 0.1% (w/w) and has been proved to possess excellent compliance better than ones with 0.1% (w/w) and 0.25% (w/w) atropine concentrations. Preferably, the range of the atropine solution is from 0.001% (w/w) to approximate 0.1% (w/w). Experiments are shown and illustrated as the following descriptions and comparison tables.

Firstly, clinic patients were observed to have photophobia and poor compliance in administration of 0.1% (w/w) atropine solution. However, clinic patients treated with 0.05% (w/w) atropine solution had less mydriasis phenomenon and photophobia and were improved in prevention of myopia progression in comparison with ones treated with 0.1% (w/w) and 0.25% (w/w) atropine solutions with references to Table 1 and Table 2.

TABLE 1
Shin's study published in 1999

| | Treatment group and control group | | | |
|---|---|---|---|---|
| Atropine | 0.5% | 0.25% | 0.1% | Control (0%) |
| concentration | | | | |
| Myopia | 0.04 | 0.45 | 0.47 | 1.06 |
| progression | (diopter/yr) | (diopter/yr) | (diopter/yr) | (diopter/yr) |

TABLE 2
study of the present invention in 2006

Treatment group and control group

| | | |
|---|---|---|
| Atropine | 0.05% | Control (0%) |
| concentration | | |
| Myopia | 0.28(diopter/yr) | 0.75(diopter/yr) |
| progression | | |

With regard to Table 2, 21 patients (42 eyes) were subject in the treatment group and 36 patients were subject in the control group. All patients are residents in southern Taiwan. Male-female ratios in the treatment group and the control group were respectively 12:9 and 18:18.

Wherein, an average age in the treatment group was 8.38±1.47 years old (range from 6 to 12 years old) and an average age in the control group was 8.11±1.12 years old (range from 6 to 12 years old).

Treating periods of the treatment group and the control group in average are respectively 19.95±9.04 months and 21.47±10.02.

According to experimental data in Tables 1 and 2, 0.05% (w/w) atropine solution has good efficiency in comparison with ones of 0.1% (w/w) and 0.25% (w/w) in myopia prevention.

Additionally, following Table 3 shows the yearly myopic statuses of patients treated with 0.05% (w/w) atropine solution.

TABLE 3
monitoring the patients treated with 0.05%(w/w)
atropine solution in myopia progression

| | 1^{st} | 2^{nd} | 3^{rd} | All (average) |
|---|---|---|---|---|
| Increased | 0.34(D/yr) | 0.32(D/yr) | 0.14(D/yr) | 0.29(D/yr) |
| diopter (D) | | | | |
| per year | | | | |

According to Table 3, the patient treated with 0.05% (w/w) atropine solution had a reduced myopia progression in yearly follow-up so that the long-term efficiency of 0.05% (w/w) atropine solution was provided to be excellent.

Moreover, the myopia exacerbation also substantially relates to visual distance during near working that the patient continuously overuses high accommodation capability of eyes. Therefore, when the accommodation capability is inhibited, the myopia does not exacerbate easily as shown in Table 4.

TABLE 4
influences on accommodation capability with administration
of different atropine concentrations

Treatment groups and control group

| | | | |
|---|---|---|---|
| Atropine | 0.05% | Control (0%)) | 0.1% |
| concentration | | | |
| Accommodation | 2.45(D) | 7.41(D) | 2.22(D) |
| capability | | | |

| | |
|---|---|
| P-value | P = 0.001 |

P = 0.001

With regard to Table 4, 21 patients were treated with 0.05% (w/w) atropine solution in a first treatment group, 9 patients were treated with 0.1% (w/w) atropine solution in a second treatment group and 6 patients were treated with blank solution in the control group. Male-female ratios in the treatment groups and the control group were respectively 12:10 in the first treatment group, 5:4 in the second treatment group and 3:3 in the control group.

Wherein, average ages were respectively 9.4±1.7 years old in the first treatment group, 11±1.7 years old in the second treatment group and 10±2 years old in the control group.

Treating periods of the treatment groups in average were respectively 22.43±12.9 months in the first treatment group and 25.88±12.47 months in the second treatment group.

Observation timing after applying the atropine solutions were respectively 16.37±2.53 hours in the first treatment group and 17.11±1.9 hours in the second treatment group.

According to experimental data in Table 4, 0.05% (w/w) atropine solution had good efficiency in comparison with ones of 0.1% (w/w) (w/w) in myopia prevention but had no significantly difference. However, the first and second testament groups did have noticeable accommodation-inhibiting efficiency in comparison with the control group because the P-value was 0.001 (less than 0.05) which represented difference in statistic. Therefore, 0.05% (w/w) atropine solution was proved to have excellent accommodation-inhibiting efficiency.

TABLE 5
mydriasis phenomenon after administration
of different atropine concentrations

| | Treatment groups and control group | | |
|---|---|---|---|
| Atropine | 0.05% | Control | 0.1% |
| concentration | | | |
| Pupil diameter | 5.52 mm | 2.93 mm | 6.10 mm |
| P-value | P < 0.0001 | | |
| | | P < 0.0001 | |
| | P = 0.0364 | ————————— | [symbol] |

According to Table 5, mydriasis phenomenon with 0.05% (w/w) atropine solution was significantly minor in comparison with one with 0.1% (w/w) atropine solution in statistics (P<005).

According to following Table 6, patients treated with 0.05% (w/w) atropine solution suffered the photophobia in a lower ratio in comparison with ones treated with 0.1% (w/w) atropine solution. Therefore, 0.05% (w/w) atropine solution significantly reduced the side-effect of photophobia.

TABLE 6
photophobia ratios with administration
of different atropine concentrations

| | | |
|---|---|---|
| Atropine | 0.05% | 0.1% |
| concentration | | |
| Photophobia ratio | 66.67% | 85.71% |

From the clinic statistics summarized from the foregoing tables, 0.1% (w/w) atropine solution certainly caused photophobia to patients and had poor compliance. However, 0.05% (w/w) atropine solution in the present invention noticeably reduced the photophobia and the mydrasis phenomenon (as shown in Tables 5 and 6) and improved in prevention of myopia progression. In comparison with 0.1% (w/w) and 0.25% (w/w) atropine solutions, 0.05% (w/w) atropine solution had the same efficiency without difference (as shown in Table 1 and Table 2) and patients did not worry about side-effects to eyes or bodies so that medicine compliance of atropine administration was thus improved to reduce treatment barrier.

In following Table 7, mydriasis phenomenon was observed to have 7 to 14 days duration in administration of 1% (w/w) atropine solution, 2 days duration in administration of cyclopentolate hydrochloride, and 6 hours duration in administration of tropicamide, wherein all three ingredients were subjected to treat myopia but only atropine was efficient. According to Table 7, mydriasis phenomenon only showed 12 to 18 hours duration in administration of 0.05% (w/w) atropine solution that had minor influence on patients in comparison with ones of 1% (w/w) atropine solutions.

TABLE 7
durations of mydriasis phenomenon in administration of
different ingredients for preventing myopia progression

| | | | Occurring | Duration of | |
|---|---|---|---|---|---|
| Common name | Registered name | Concentration (%) | time | mydriasis | |
| Atropine sulfate | Atropisol | Soln, 0.5%-2% | 45-120 | 7-14 | days |
| | Atropine-Care | Soln, 1% | minutes | | |
| | Isopto Atropine | Soln, 0.5%-3% | | | |
| | Available generically | Soln, 1% | | | |
| | | Ointment, 0.5%-1% | | | |
| Cyclopentolate | AK-Pentolate | Soln, 1% | 30-60 | 2 | days |
| Hydrochloride | Cycogyl | Soln, 0.5%-2% | minutes | | |
| | Available generically | Soln, 1% | | | |
| Tropicamide | Mydriacyl | Soln, 0.5%, 1% | 20-40 | 4-6 | days |
| | Tropicacyl | Soln, 0.5%, 1% | minutes | | |
| | Available generically | Soln, 0.5%, 1% | | | |
| Atropine sulfate | | Soln, 0.05% | 45-120 | 12-18 | hours |
| | | | minutes | | |

With regard to a preparing method for the low-concentration atropine solution in the present invention preferably is to dilute atropine ingredient with distilled water or saline to obtain atropine concentration less than 0.1% (w/w). Preferably, the atropine concentration is 0.05% (w/w).

The low-concentration atropine solution for preventing myopia progression has the following advantages:

1. Mydriasis phenomenon is reduced to avoid photophobia, increase medication compliance and reduce dropout rate during treatment.

2. The low-concentration atropine solution efficiently depresses eye accommodation to prevent myopia progression.

3. The low-concentration atropine solution causes no systemic side-effect to patients.

4. The low-concentration atropine solution has no rapidly rebounding myopia progression as caused by the high-concentration atropine solution after quitting the administration.

Although this invention has been described in its preferred form with a certain degree of particularity, it is understood that the present invention of the preferred form has been made only by way of example and that numerous changes in the details of construction and the combination and arrangement of parts any be resorted to without departing from the spirit and scope of the invention.

What is claimed is:

1. A low-concentration atropine solution for preventing myopia progression comprising:
atropine concentration less than 0.1% (w/w).

2. The low-concentration atropine solution as claimed in claim 1, wherein the atropine concentration is 0.05% (w/w).

3. The low-concentration atropine solution as claimed in claim 1, wherein the atropine concentration has a range from 0.001% (w/w) to approximate 0.1% (w/w).

4. A preparing method of the low-concentration atropine solution for preventing myopia progression is to dilute an atropine ingredient to obtain an atropine concentration less than 0.1% (w/w).

5. The preparing method of the low-concentration atropine solution as claimed in claim 4, wherein the atropine ingredient is diluted with distilled water.

6. The preparing method of the low-concentration atropine solution as claimed in claim 4, wherein the atropine ingredient is diluted with saline.
